(19) United States
(12) Patent Application Publication
(10) Pub. No.: US 2017/0137491 A1
(43) Pub. Date: May 18, 2017

(54) Novel Method

(71) Applicant: Cambridge Enterprise Limited, Cambridge, Cambridgeshire (GB)

(72) Inventors: Nicholas MATHESON, Cambridge (GB); Paul LEHNER, Cambridge (GB); Andrew PEDEN, Cambridge (GB)

(73) Assignee: Cambridge Enterprise Limited, Cambridge, Cambridgeshire (GB)

(21) Appl. No.: 15/317,777

(22) PCT Filed: Jun. 10, 2015
(86) PCT No.: PCT/GB2015/051694

§ 371 Date: Dec. 9, 2016

(30) Foreign Application Priority Data
Jun. 10, 2014 (GB) 1410262.8

(51) Int. Cl. C07K 14/71; G01N 33/569

(52) U.S. Cl. CPC C07K 14/71; G01N 33/56966; C07K 2319/70; C07K 2319/03

(57) ABSTRACT

The invention relates to a method of cell selection by using a nucleic acid molecule comprising a first nucleic acid sequence encoding a streptavidin binding peptide and a second nucleic acid sequence encoding a cell surface protein. The invention also relates to nucleic acid molecules, vectors, cells and kits for use with said method.

### FIELD OF THE INVENTION

The invention relates to a method of cell selection by using a nucleic acid molecule comprising a first nucleic acid sequence encoding a streptavidin binding peptide and a second nucleic acid sequence encoding a cell surface protein. The invention also relates to nucleic acid molecules, vectors, cells and kits for use with said method.

### BACKGROUND OF THE INVENTION

Pure populations of transfected or transduced mammalian cells are commonly isolated from mixed samples by co-expression of a gene or short hairpin RNA (shRNA) of interest with three sorts of phenotypic marker: an exogenous gene encoding drug or antibiotic resistance; an internal fluorescent protein, such as Green Fluorescent Protein (GFP), enabling Fluorescence-Activated Cell Sorting (FACS); or a cell surface protein combined with antibody labelling. Where antibody labelling of a cell surface marker is used, antibodies may be either conjugated to a fluorochrome for FACS, or to biotin for affinity purification using a solid streptavidin-conjugated matrix, typically magnetic beads (Dainiak, Kumar et al. (2007) Adv. Biochem. Eng. Biotechnol. 106: 1-18). Compared with FACS, immunomagnetic selection is relatively fast, simple and scalable for simultaneous processing of multiple samples and large cell numbers (Miltenyi, Muller et al. (1990) Cytometry 11(2): 231-238; Dainiak, Kumar et al. (2007) Adv. Biochem. Eng. Biotechnol. 106: 1-18). It is supported by a number of widely used commercial systems (Neurauter, Bonyhadi et al. (2007) Adv. Biochem. Eng. Biotechnol. 106: 41-73; Grutzkau and Radbruch (2010) Cytometry 77(7): 643-647) including specific product lines for the enrichment of cells using exogenous CD4, H-2k or LNGFR (MACSelect; Miltenyi, Muller et al. (1990) Cytometry 11(2): 231-238) or a membrane-targeted mCherry fusion protein (CherryPicker™; Clontech) as the cell surface marker for antibody labelling.

Following immunomagnetic selection, cells typically remain coated with magnetic beads and antibody-antigen complexes, risking alteration of their behaviour or viability through cross-linking of cell-surface receptors (triggering signalling) or internalisation of the ferrous beads (leading to toxicity) (Neurauter, Bonyhadi et al. (2007) Adv. Biochem. Eng. Biotechnol. 106: 41-73). Methods have therefore been devised to release the beads through use of a low affinity biotin, cleavage of a nucleic acid linker, or competition with a selected Fab (antigen-binding) antibody fragment (Neurauter, Bonyhadi et al. (2007) Adv. Biochem. Eng. Biotechnol. 106: 41-73). These approaches are limited, however, by requirements for additional individualized reagents and/or leave cells coated with residual antibody-antigen complexes.

Examples of commercially available systems which release positively selected cells from magnetic beads include: Dynabeads FlowComp, CELLection Biotin Binder and DETACHaBEAD (all provided by Invitrogen). All of these techniques rely on the use of antibodies and therefore suffer from limitations to do with the availability and cost of specific antibodies, as well as the difficulty in trying to remove antibodies from the selected cells after bead release.

A system for direct (antibody-free) isolation of “untouched” cells (i.e. cells not coated in antibodies and/or antibody-antigen complexes) using magnetic beads must address two requirements:
(i) a high affinity receptor-ligand interaction, wherein the ligand may be expressed at the cell surface and the receptor may be immobilised on the beads; and
(ii) a method to subsequently break this receptor-ligand interaction, following selection, to release the cells from the beads.

High-affinity streptavidin-binding peptides (e.g. Nano-tags where K_{d} for streptavidin is less than 20 nM) have recently been described (see Keefe, Wilson et al. (2001) Protein Expr. Purif. 23(3): 440-446; Wilson, Keefe et al. (2001) PNAS 98(7): 3750-3755; Lamla and Erdmann (2004) Protein Expr. Purif. 33(1): 39-47) and, in combination with immobilised streptavidin, fulfil these requirements. Critically, whilst their high affinity for streptavidin may facilitate efficient bead-based magnetic selection (requirement (i)), cells may nonetheless subsequently be released through competition with biotin (requirement (ii)).

Conversely, intermediate-affinity biotin-mimetic peptides (BMPs; K_{d} for streptavidin greater than 200 nM) have been known for decades (see Geibel et al. (1995) Biochemistry 34(47): 15430-15435). WO 2012/085911 describes nucleic acid molecules comprising a nucleic acid sequence encoding such a BMP or biotin acceptor peptide (BAP), suitable for use in fluorescence-activated cell sorting (FACS) of cells which therefore remained coated in BMP-streptavidin complexes (see Heiman et al. (2014) Cytometry A 85(2): 162-168).

There is therefore a need to develop an improved method of cell sorting which overcomes the problems associated with current techniques.

### SUMMARY OF THE INVENTION

According to a first aspect of the invention, there is provided a method of cell selection comprising:
(a) transfecting or transducing a cell with a nucleic acid molecule comprising a first nucleic acid sequence encoding a streptavidin binding peptide and a second nucleic acid sequence encoding a cell surface protein;
(b) expressing the nucleic acid in the cell;
(c) isolating the cell using streptavidin linked to a solid matrix; and
(d) removing the cell from the solid matrix using biotin.

According to a further aspect of the invention, there is provided a nucleic acid molecule comprising a first nucleic acid sequence encoding the Streptavidin Binding Peptide of SEQ ID NO: 1 and a second nucleic acid sequence encoding a cell surface protein.

According to a further aspect of the invention, there is provided a vector comprising a nucleic acid molecule as defined herein.

According to a further aspect of the invention, there is provided a host cell which contains the vector as defined herein.

According to a further aspect of the invention, there is provided a cell selection kit comprising the vector as defined herein and optionally together with instructions to use said kit in accordance with the method defined herein.

According to a further aspect of the invention, there is provided the use of a kit for selecting cells comprising a vector as defined herein.

### BRIEF DESCRIPTION OF THE FIGURES

FIGS. 1A-1D: SBP-ΔLNGFR cell surface affinity tag for Antibody-Free Magnetic Cell Sorting. In Antibody-Free Magnetic Cell Sorting (FIG. 1A) transfected or transduced cells co-express a gene or shRNA of interest with a streptavidin-binding cell surface affinity tag. Cells are selected by incubation with streptavidin-conjugated beads then, after washing to remove unbound cells, released by incubation with excess biotin. SBP-ΔLNGFR comprises the 38 amino acid SBP fused to the N-terminus of the truncated LNGFR (FIG. 1B). Expression of SBP-ΔLNGFR at the cell surface was tested 48 hours after transient transfection of 293 Ts with pHRSIN-HA-SBP-ΔLNGFR by staining with streptavidin-APC (FIG. 1C). After a further 72 hours, cells expressing SBP-ΔLNGFR were selected from the bulk population using magnetic streptavidin-conjugated beads: (i) Dynabeads Biotin Binder (Invitrogen) or (ii) Streptavidin MicroBeads (Miltenyi) (FIG. 1D). Purity of transfected cells before (dotted line) and after (grey and black lines) selection was assessed by staining with anti-LNGFR-PE. Background staining of cells transfected with a control vector is shown (light grey shading).

FIGS. 2A-2D: Phenotypic selection using SBP-ΔLNGFR. 293 Ts were transiently transfected or lentivirally transduced with pHRSIN-SE-PGK-SBP-ΔLNGFR-W (encoding EGFP and SBP-ΔLNGFR; FIG. 2A) or pHRSIREN/β2m-PGK-SBP-ΔLNGFR-W (encoding shRNA to β2m and SBP-ΔLNGFR; FIG. 2B) and stained with streptavidin-APC plus/minus anti-HLA-A2-PE. Transfected/transduced cells are either GFP+/streptavidin-APC+ or HLA-A2-low/streptavidin-APC+(dashed circles). Primary human CD4+ T cells were lentivirally transduced with the same constructs then selected using Dynabeads Biotin Binder. Purity of cells before (grey line) and after (black line) selection was assessed by GFP fluorescence (FIG. 2C) or staining with anti-HLA-A2-PE (FIG. 2D). Transduced cells are either GFP+ or HLA-A2-low (dashed boxes). Background staining of untransfected/unstransduced controls is shown (light grey shading).

FIGS. 3A-3D: Optimised Antibody Free Magnetic Cell Sorting of primary human CD4+ T cells. Primary human CD4+ T cells were lentivirally transduced with pHRSIREN/β2m-PGK-SBP-ΔLNGFR-W (encoding shRNA to β2m and SBP-ΔLNGFR under the PGK promoter) and either rested for 2 weeks (grey line) or re-stimulated with CD3/CD28 Dynabeads 3 days prior to analysis (black line). Cells were co-stained with anti-HLA-A2-PE and anti-LNGFR-APC, and expression levels of SBP-ΔLNGFR compared in HLA-A2-low cells (FIG. 3A). Transduction with pHRSIN-SE-PGK-SBP-ΔLNGFR-W was then compared with pHRSIN-SE-P2A-SBP-ΔLNGFR-W (encoding GFP-P2A-SBP-ΔLNGFR under the spleen focus-forming virus (SFFV) promoter) (FIG. 3B). Transduced cells are GFP+/LNGFR-APC+(dashed circles). Background staining of untransfected/unstransduced controls is shown (light grey shading). Finally, primary human CD4+T cells were transduced with the optimised pHRSIREN-S-SBP-ΔLNGFR-W and pHRSIN-SE-P2A-SBP-ΔLNGFR-W lentivectors (FIG. 3C) encoding 2 different shRNAs and 2 different exogenous genes. Following selection with Dynabeads Biotin Binder purity was assessed by staining with anti-LNGFR-PE (FIG. 3D). Each datapoint represents % LNGFR+ for a different construct (shRNA or exogenous gene) and means and SEMs are shown. cPPT—central polypurine tract; RRE—Rev response element; ψ—packaging signal; LTR—long terminal repeat; WPRE—Woodchuck Hepatitis Virus post-transcriptional regulatory element.

FIG. 4: Antibody-Free Magnetic Cell Sorting of 293T cells following CRISPR/Cas9 genome editing. 293 Ts were transiently transfected with pSpCas9(BB)-P2A-SBP-ΔLNGFR (encoding gRNA to β2m and Cas9-P2A-SBP-ΔLNGFR) and stained with anti-MHC-I-AF647 before (grey line) or after (black line) selection with Dynabeads Biotin Binder. Transfected cells with β2m knockouts are MHC-I low (dashed boxes). Background staining of un-transfected controls is shown (light grey shading).

FIG. 5: Codon-optimised SBP-ΔLNGFR construct. DNA and amino acid sequences of the codon-optimised SBP-ΔLNGFR construct in pHRSIN-SE-P2A-SBP-ΔLNGFR-W are shown. BamHI and NotI sites may be used to insert the gene of interest (without stop codon) upstream of the P2A peptide for co-translation with SBP-ΔLNGFR. In pHRSIREN-S-SBP-ΔLNGFR-W, the coding sequence starts with the murine immunoglobuin signal peptide (*) and the shRNA of interest is inserted separately in the U6-shRNA cassette using BamHI and EcoRI sites. In pSpCas9(BB)-P2A-SBP-ΔLNGFR, the Cas9 nuclease is located upstream of the P2A peptide for co-translation with SBP-ΔLNGFR and the gRNA of interest is inserted separately in the U6-gRNA cassette using BbsI sites. Locations of ribosomal skipping (†) and signal peptidase (‡) cleavage are shown. Following signal peptidase cleavage the construct is anchored to the plasma membrane by the transmembrane region (TM) of the truncated LNGFR. Unshaded amino acids comprise flexible linker regions. Assembly is modular and unique restriction sites are highlighted.

### DETAILED DESCRIPTION OF THE INVENTION

### Methods of Cell Selection

According to a first aspect of the invention, there is provided a method of cell selection comprising:
(a) transfecting or transducing a cell with a nucleic acid molecule comprising a first nucleic acid sequence encoding a streptavidin binding peptide and a second nucleic acid sequence encoding a cell surface protein;
(b) expressing the nucleic acid in the cell;
(c) isolating the cell using streptavidin linked to a solid matrix; and
(d) removing the cell from the solid matrix using biotin.

The method described herein provides the use of a cell surface streptavidin binding peptide for magnetic cell sorting, combining the advantages of bead-based cell isolation with the ability to release beads from selected cells by competition with biotin. This method allows for “marker-free” selection (in particular, selection without the need for antibodies) which simplifies the process of cell selection and overcomes the disadvantages associated with current methodologies, such as the availability and cost of specific reagents and antibodies, as well as the difficulty in trying to remove antibodies after cell selection.

Further advantages with the described method include that no deficit in cell viability or function in a wide range of downstream applications has been observed, and the method may be completed (including multiple samples) extremely quickly, for example in less than 1 hour. In particular, it was found that bound cells could be completely released from streptavidin-conjugated beads by incubation with 2 mM biotin for as little as 15 minutes.

References herein to “transfection” and “transduction” refer to methods in which target DNA is deliberately introduced into a cell. Methods of transfection and transduction are well known in the art, for example by chemical means (e.g. calcium phosphate, cationic polymers or liposomes) or non-chemical means (e.g. electroporation, sonoporation, optical transfection, spinoculation, a gene gun, magnetic-assisted transfection, impalefection, viral transduction). In one embodiment, step (a) is performed by transfection. In an alternative embodiment, step (a) is performed by transduction.

In one embodiment, step (a) is performed using a transfection reagent, for example a reagent selected from FuGENE 6 (Promega) or TranIT-293 (Mirus). In an alternative embodiment, step (a) is performed by spinoculation (i.e. wherein the vector is introduced into the cell using centrifugal forces).

In one embodiment, more than 0.5 mM biotin is used to release the cells from the solid matrix, such as more than 1 mM, 1.5 mM or 2 mM. In a further embodiment, about 2 mM biotin is used to release the cells from the solid matrix.

References herein to “biotin” refer to the naturally occurring vitamin, also known as vitamin H. It is a molecule well-known in the art, especially in the field of biotechnology where biotin is frequently used to isolate proteins in biochemical assays which involve its binding partner streptavidin. It will be understood that references to the term “biotin” include naturally occurring and synthetically made biotin.

An advantage of the method described herein is that the naturally occurring vitamin, biotin, can be used to release the selected cells by outcompeting the streptavidin binding peptide bound to the streptavidin coated solid matrix. This means that a non-toxic, cheap and widely available means can be used to elute the selected cells from the solid matrix.

In one embodiment, the cells are removed from the solid matrix using a release buffer (RB) which comprises complete media (e.g. RPMI-1640 with 10% FCS and 1% penicillin/streptomycin), 10 mM HEPES buffer and 2 mM biotin. In a further embodiment, the release buffer is at pH 7.4.

In one embodiment, the method additionally comprises a washing step after step (c) to remove any cells which have not bound to the solid matrix (i.e. unsuccessfully transfected/transduced cells). This step helps to purify the selected cells further. In a further embodiment, the wash step uses an incubation buffer (IB) which comprises PBS without calcium/magnesium, 2 mM EDTA and 0.1% BSA. In a yet further embodiment, the incubation buffer is at pH 7.4.

### Cell Selection and Uses

In one embodiment, the solid matrix is selected from beads (e.g. magnetic beads) or a membrane (e.g. glass or nitrocellulose). It will be understood that the solid matrix is coated with streptavidin to allow for the selection of successfully transfected/transduced cells which will express the streptavidin binding peptide on the cell surface.

In a further embodiment, the solid matrix comprises magnetic beads. The use of a solid matrix, such as the use of magnetic beads, has many advantages, for example beads are cheap, easy to setup and scalable and are much quicker to use than FACS methods.

The methods and vectors described herein may be used throughout the field of biotechnology, wherever marker free cell selection is required. One particular use of the present method is in CRISPR (Clustered Regularly Interspaced Short Palindromic Repeats)-Cas9 gene editing (Ran, Hsu et al. (2013) Nat. Protoc. 8(11): 2281-2308; Shalem, Sanjana et al. (2014) Science 343(6166)): 84-87). CRISPR is a method of selectively knocking out genes, but there have been difficulties in selecting the cells with the gene knock-out, therefore the present method may be used to isolate these cells.

Another use of the present method is to use the streptavidin binding peptide-cell surface protein construct as a reporter gene for selection of cells in which a promoter of interest is active in vitro or in vivo.

### Nucleic Acid Molecules

According to a further aspect of the invention, there is provided a nucleic acid molecule comprising a first nucleic acid sequence encoding the Streptavidin Binding Peptide of SEQ ID NO: 1 and a second nucleic acid sequence encoding a cell surface protein.

The isolated nucleic acid molecules and vectors described herein provide a way for a streptavidin binding peptide to be expressed on the cell surface for use in methods of cell sorting. This allows for “marker-free” selection (in particular, selection without the need for antibodies) which simplifies the process of cell selection and overcomes the disadvantages associated with current methodologies, such as the availability and cost of specific reagents and antibodies, as well as the difficulty in trying to remove antibodies after cell selection.

In particular, the nucleic acid molecules and vectors may be used in methods of magnetic cell sorting and combines the advantages of bead-based cell isolation with the ability to release beads from selected cells by competition with biotin.

References herein to a “streptavidin binding peptide” or “SBP” refer to a peptide which is able to bind streptavidin, although with less binding efficiency than biotin. In one embodiment, the dissociation constant (K_{d}) of the SBP for streptavidin is less than 20 nM, such as less than 15 nM or less than 10 nM.

In one embodiment, the SBP described herein comprises the amino acid sequence of SEQ ID NO: 1:

(SEQ ID NO: 1)
5′-MDEKTTGWRGGHVVEGLAGELEQLRARLEHHPQGQREP-3′

In a further embodiment, the SBP described herein comprises the nucleic acid sequence of SEQ ID NO: 2:

(SEQ ID NO: 2)
5′-atggacgaaaagaccacaggatggcgaggaggacacgtggtcgaggg
actggcaggagagctggaacagctgcgggctagactggaacaccatcctc
agggacagcgagagcca-3′

Streptavidin-binding peptide tags with nanomolar dissociation constants for streptavidin have been generated for the purification of recombinant proteins (see Keefe, Wilson et al. (2001) Protein Expr. Purif. 23(3): 440-446; Wilson, Keefe et al. (2001) PNAS 98(7): 3750-3755; Lamla and Erdmann (2004) Protein Expr. Purif. 33(1): 39-47). The present inventors have found that expression of a cell surface Streptavidin Binding Peptide tag of SEQ ID NO: 1 can be used to select cells co-expressing a gene or shRNA of interest by binding directly to streptavidin beads, without the need for antibody labelling. Furthermore, selected cells could subsequently be released from the beads by incubation with biotin, a naturally occurring vitamin already present in many cell culture media, leaving cells free of antibody and beads.

Other examples of suitable streptavidin binding peptides include Nano-tags, such as those described in Lamla and Erdmann (2004) Protein Expr. Purif. 33(1): 39-47, or the proteins described in Wilson, Keefe et al. (2001) PNAS 98(7): 3750-3755. The sequences of these SBPs are listed in Table 1:

TABLE 1
Examples of streptavidin binding peptides

| | SEQ |
|---|---|
| | ID |
| Sequence | NO. |
| DVEAWLDERVPLVET | 3 |
| DVEAWLGERVPLVET | 4 |
| DVEAWLGARVPLVET | 5 |
| DVEAWLDER | 6 |
| DVEAWLGER | 7 |
| DVEAWLGAR | 8 |
| MDEKTHCFHPGDHLVRLVEELQALAEGLQRQGGRQPHRLPRRRPH | 9 |
| HLQLLLDEAHPQAGPLRERAHQVDGRLLLQHHPQGDRLLQQPQDH | |
| PLELVWRLPPS | |
| MDEKTHCTISMNGAVPLVPHHHPQGDPLRLLHRPQPALLVRHPQG | 10 |
| DLVALVEHHEGVDRGLVALPELHAEELGEPVGDLVQGPVEQVQGV | |
| VDALVWRLPPS | |
| MDEKTHWVNVYHPQGDLLVRGHGHDVEALHDQGLHQLDLLVGPPP | 11 |
| EVVRALRGEVLGGLRRLVPLDHPQGEALDQARQRPQHLLELHHRA | |
| LPPALVWRLPPS | |
| MDEKTHWLEDLKGVLKDCLKDLMDFTKDCRSPRVQPQPLLHHDRG | 12 |
| EPVPLLREAGRDLGGLGPRAPRQARPLHHGRHDLHEPLVLQDHPQ | |
| GGPLVCGCHHH | |

Therefore, in one embodiment, the SBP described herein comprises an amino acid sequence selected from any one of SEQ ID NOs: 1, 3, 4, 5, 6, 7, 8, 9, 10, 11 or 12, or fragments or variants thereof.

It will be understood that references to a “variant” refer to a sequence which is related to a sequence described herein. Such variants may differ from the sequences disclosed herein by 1, 2, 3, 4 or 5 amino acids, such as 1 or 2 amino acids, in particular 1 amino acid.

It will be understood that references to a “fragment” refer to a portion of a sequence described herein. For example, a fragment may comprise a C-terminal truncation, or a N-terminal truncation. Fragments are suitably greater than 4 amino acids in length, for example 5, 6, 7, 8, 9, 10, 11, 12, 13, 14, 15, 16, 17, 18, 19, or 20 amino acids in length.

References herein to “cell surface protein” (or “membrane associated carrier peptide”) refer to a protein which is expressed on the cell surface. It will be understood that this term includes the use of cell surface peptides. This protein/peptide enables the streptavidin binding peptide to be expressed on the cell surface so that the cell can be isolated using a streptavidin coated matrix.

In one embodiment, the cell surface protein is a non-functional protein. The use of a non-functional protein prevents the protein from being involved in cell signalling which may affect the expression of the streptavidin binding peptide on the cell surface. It will be apparent to the person skilled in the art that there are several ways to make the protein “non-functional” (i.e. inactive), for example, by removing the signalling domain of the protein, such as the cytoplasmic domain of the protein.

In an alternative embodiment, the cell surface protein is a functional protein.

In one embodiment, the cell surface protein is selected from: Low-Affinity Nerve Growth Factor Receptor (LNGFR), CD4, H-2K, CherryPicker™ or phOx sFv.

In a further embodiment, the cell surface protein is a non-functional Low-Affinity Nerve Growth Factor Receptor (LNGFR). LNGFR is a 399 amino acid Type I transmembrane cell surface glycoprotein member of the Tumour Necrosis Factor Receptor superfamily (Rogers, Beare et al. (2008) J. Biol. Regul. Homeost. Agents 22(1): 1-6). An example of a non-functional LNGFR is where the cytoplasmic domain of the LNGFR has been removed to form a truncated LNGFR.

The Examples described herein show that a high level of cell surface streptavidin-binding peptide expression was achieved using this cell surface protein as a successful carrier.

In an alternative embodiment, the cell surface protein is CD4. In an alternative embodiment, the cell surface protein is H-2K (a mouse MHC class I protein).

In an alternative embodiment, the cell surface protein is CherryPicker™′ CherryPicker™ is a membrane-targeted red fluorescent protein which has previously been used in the Clontech CherryPicker™ Cell Capture (IRES) Vector Set.

In an alternative embodiment, the cell surface protein is phOx sFv. This membrane-anchored single chain antibody (sFv) is directed against phOx (hapten 4-ethoxymethylene-2-phenyl-2-oxazolin-5-one) and has previously been used in the Invitrogen Capture-Tec™ pHook™-3 System.

In one embodiment, the non-functional protein comprises an amino acid sequence of SEQ ID NO: 13:

(SEQ ID NO. 13)
5′-KEACPTGLYTHSGECCKACNLGEGVAQPCGANQTVCEPCLDSVTFSD
VVSATEPCKPCTECVGLQSMSAPCVEADDAVCRCAYGYYQDETTGRCEAC
RVCEAGSGLVFSCQDKQNTVCEECPDGTYSDEANHVDPCLPCTVCEDTER
QLRECTRWADAECEEIPGRWITRSTPPEGSDSTAPSTQEPEAPPEQDLIA
STVAGVVTTVMGSSQPVVTRGTTDNLIPVYCSILAAVVVGLVAYIAFK
R-3′

This sequence encodes a truncated Low-Affinity Nerve Growth Factor Receptor peptide (ΔLNGFR) used in the Examples described herein. The data shows that this non-functional protein may be coupled with a streptavidin binding peptide and successfully expressed on the cell surface.

In one embodiment, the nucleic acid molecule encodes the amino acid sequence of SEQ ID NO: 14:

(SEQ ID NO. 14)
5′-MDEKTTGWRGGHVVEGLAGELEQLRARLEHHPQGQREPGSGAIAKEA
CPTGLYTHSGECCKACNLGEGVAQPCGANQTVCEPCLDSVTFSDVVSATE
PCKPCTECVGLQSMSAPCVEADDAVCRCAYGYYQDETTGRCEACRVCEAG
SGLVFSCQDKQNTVCEECPDGTYSDEANHVDPCLPCTVCEDTERQLRECT
RWADAECEEIPGRWITRSTPPEGSDSTAPSTQEPEAPPEQDLIASTVAGV
VTTVMGSSQPVVTRGTTDNLIPVYCSILAAVVVGLVAYIAFKR-3′

This sequence encodes the Streptavidin Binding Peptide (SBP) tag fused to the N-terminus of the truncated Low-Affinity Nerve Growth Factor Receptor peptide (ΔLNGFR). As shown in the Examples described herein, this construct enabled successfully transfected/transduced cells to express the streptavidin binding peptide on their cell surface so that they could be easily isolated during cell selection. Furthermore, this construct enabled the expression of the streptavidin binding tag on the cell surface without any disruption to cell viability or function.

In one embodiment, the nucleic acid molecule additionally comprises a signal peptide. In one embodiment, the signal peptide is selected from a murine immunoglobulin or a LNGFR signal peptide. In a further embodiment, the signal peptide encodes the amino acid sequence of SEQ ID NO: 15:

(SEQ ID NO: 15)
5′-MGWSCIILFLVATATGVHSQVQ-3′

In one embodiment, the cell surface protein is a Type I transmembrane protein. It will be understood by a person skilled in the art that in this embodiment the SBP would need to be inserted after the signal peptide, but before the cell surface protein, in order for the SBP to be expressed at the cell surface.

In an alternative embodiment, the cell surface protein is a Type 2 transmembrane protein. In yet further embodiment, the cell surface protein is a multi-pass polytopic transmembrane protein. In these embodiments, the SBP is fused to the extracellular C-terminus of the cell surface protein, to ensure expression of the SBP at the cell surface.

Therefore, in one embodiment, the nucleic acid molecule encodes the amino acid sequence of SEQ ID NO: 16:

(SEQ ID NO. 16)
5′-MGWSCIILFLVATATGVHSQVQLEGSGMDEKTTGWRGGHVVEGLAGE
LEQLRARLEHHPQGQREPGSGAIAKEACPTGLYTHSGECCKACNLGEGVA
QPCGANQTVCEPCLDSVTFSDVVSATEPCKPCTECVGLQSMSAPCVEADD
AVCRCAYGYYQDETTGRCEACRVCEAGSGLVFSCQDKQNTVCEECPDGTY
SDEANHVDPCLPCTVCEDTERQLRECTRWADAECEEIPGRWITRSTPPEG
SDSTAPSTQEPEAPPEQDLIASTVAGVVTTVMGSSQPVVTRGTTDNLIPV
YCSILAAVVVGLVAYIAFKR-3′

This sequence encodes the complete SBP-ΔLNGFR amino acid sequence including a signal peptide (LNGFR is a Type 1 transmembrane protein). As shown in the Examples described herein, this construct enabled successfully transfected/transduced cells to express the streptavidin binding peptide on their cell surface so that they could be easily isolated during cell selection.

In one embodiment, the nucleic acid molecule encodes the amino acid sequence of SEQ ID NO: 17:

(SEQ ID NO. 17)
5′-AAAGSGATNFSLLKQAGDVEENPGPMGWSCIILFLVATATGVHSQVQ
LEGSGMDEKTTGWRGGHVVEGLAGELEQLRARLEHHPQGQREPGSGAIAK
EACPTGLYTHSGECCKACNLGEGVAQPCGANQTVCEPCLDSVTFSDVVSA
TEPCKPCTECVGLQSMSAPCVEADDAVCRCAYGYYQDETTGRCEACRVCE
AGSGLVFSCQDKQNTVCEECPDGTYSDEANHVDPCLPCTVCEDTERQLRE
CTRWADAECEEIPGRWITRSTPPEGSDSTAPSTQEPEAPPEQDLIASTVA
GVVTTVMGSSQPVVTRGTTDNLIPVYCSILAAVVVGLVAYIAFKR-3′

This sequence encodes the complete SBP-ΔLNGFR amino acid sequence including amino acids corresponding to a 5′ NotI cloning site (with a short linker) plus P2A peptide for co-translation with a preceding gene of interest (or the Cas9 nuclease) plus a signal peptide. As shown in the Examples described herein, this construct enabled successfully transfected/transduced cells to express the streptavidin binding peptide on their cell surface so that they could be easily isolated during cell selection.

In one embodiment, the nucleic acid molecule expressed at the cell surface encodes the amino acid sequence of SEQ ID NO: 18:

(SEQ ID NO. 18)
5′-QVQLEGSGMDEKTTGWRGGHVVEGLAGELEQLRARLEHHPQGQREPG
SGAIAKEACPTGLYTHSGECCKACNLGEGVAQPCGANQTVCEPCLDSVTF
SDVVSATEPCKPCTECVGLQSMSAPCVEADDAVCRCAYGYYQDETTGRCE
ACRVCEAGSGLVFSCQDKQNTVCEECPDGTYSDEANHVDPCLPCTVCEDT
ERQLRECTRWADAECEEIPGRWITRSTPPEGSDSTAPSTQEPEAPPEQDL
IASTVAGVVTTVMGSSQPVVTRGTTDNLIPVYCSILAAVVVGLVAYIAFK
R-3′

This sequence encodes the SBP-ΔLNGFR amino acid sequence following cleavage of the signal peptide by signal peptidase, i.e. the remaining protein expressed at the cell surface. As shown in the Examples described herein, this construct enabled successfully transfected/transduced cells to express the streptavidin binding peptide on their cell surface so that they could be easily isolated during cell selection.

In one embodiment, the nucleic acid molecule additionally comprises a promoter (i.e. operably linked to the nucleic acid sequence encoding a streptavidin binding peptide and a cell surface protein). The use of a promoter allows for the controlled or constitutive expression of the construct encoded by a nucleic acid, for example in a vector. In one embodiment, the promoter is constitutively active (i.e. leading to constitutive expression of the construct). In an alternative embodiment, the promoter is inducible (i.e. leading to controlled expression of the construct).

In one embodiment, the promoter is selected from a spleen focus-forming virus (SFFV) or phosphoglycerate kinase (PGK) promoter. In a yet further embodiment, the promoter is the SFFV promoter.

In an alternative embodiment, the nucleic acid molecule does not comprise a promoter. In this embodiment, the nucleic acid molecule is inserted into the cell genome under a native promoter. Therefore, the method described herein may be used for the selection of cells to determine if a promoter of interest (i.e. the native promoter) is active in vitro or in vivo. For example, the nucleic acid molecule (e.g. SBP-ΔLNGFR) may be used as a reporter gene so that if the promoter of interest is active then the cells can be selected by binding the SBP expressed on the cell surface.

It will be understood that references to a “native promoter” refer to a promoter which occurs naturally in the cell's genome.

In one embodiment, the nucleic acid molecule additionally comprises a distal Woodchuck Hepatitis Virus post-transcriptional regulatory element (WPRE) sequence. This sequence is used in molecular biology to increase the expression of genes introduced by viral vectors and was shown to improve construct expression in the Examples described herein.

According to a further aspect of the invention, there is provided a vector comprising a nucleic acid molecule as defined herein.

In one embodiment, the vector comprises a viral vector. In a further embodiment, the viral vector is a lentivirus vector. Lentiviruses are a subclass of retroviruses that are able to integrate DNA into the genome of non-dividing cells which makes them particularly useful in methods of molecular biology.

In one embodiment, the vector additionally comprises restriction enzyme sites suitable for insertion of a target gene. This would allow users to select for cells which they know will also contain the target gene (i.e. a gene of interest).

References herein to “restriction enzyme sites” refer to specific recognition nucleic acid sequences which are cut by restriction enzymes. Different restrictions enzymes may be chosen depending on what type of cleavage is required (e.g. to produce “sticky” or “blunt” ends). Examples of restriction enzymes include: EcoRI, EcoRII, BamHI, HindIII, TaqI, NotI, HinfI, Sau3A, PvuII, Smal, HaeII, HgaI, AluI, EcoRV, PstI, ScaI, SpeI, XhoI or XbaI. In one embodiment, the vector includes EcoRI, XhoI, NotI or BamHI restriction enzyme sites.

In one embodiment, the vector additionally comprises a target gene or short hairpin RNA (shRNA). In a further embodiment, the vector additionally comprises a target gene. In an alternative embodiment, the vector additionally comprises a short hairpin RNA (shRNA). In molecular biology, shRNA is used to silence target gene expression via RNA interference (RNAi) therefore this would allow users to select for cells which they know will contain the silenced gene target.

### Transfected and Transduced Cells

According to a further aspect of the invention, there is provided a host cell which contains the vector as defined herein.

In one embodiment, the cell is a mammalian cell, such as a human embryo kidney (HEK) cell, human T cell, Chinese Hamster Ovary (CHO) cell, baby mouse myeloma NSO cells, hamster kidney (BHK) cell, human retinal cell, COS cell, SP2/0 cell, WD 8 cell, MRCS cell or Per.C6 cells.

In a further embodiment, the mammalian cell is a human cell. In a yet further embodiment, the human cell is selected from a human embryo kidney (HEK) cell, for example HEK 293T cells, a human T cell, such as a primary human CD4⁺ T cell.

In one embodiment, the cell is a non-mammalian cell, such as a yeast, insect or plant cell.

### Kits

According to a further aspect of the invention, there is provided a cell selection kit comprising the vector as defined herein and optionally together with instructions to use said kit in accordance with the method defined herein.

In one embodiment, the kit additionally comprises one or more components selected from: streptavidin coated magnetic beads, biotin, release buffer and wash buffer, such as incubation buffer. In a further embodiment, the kit additionally comprises streptavidin coated magnetic beads, optionally together with a magnet.

Suitably a kit according to the invention may contain one or more additional components selected from: one or more controls, one or more reagents and one or more consumables.

According to a further aspect of the invention, there is provided the use of a kit for selecting cells comprising a vector as defined herein.

The invention will now be described in relation to the following Examples:

### Example 1: Development of Antibody-Free Magnetic Cell Sorting

### Materials and Methods

### Antibodies and Reagents

The following fluorescent conjugates were used for flow cytometry: ME20.4 anti-LNGFR-PE/APC (BioLegend); BB7.2 anti-HLA-A2-PE (BioLegend); W6/32 anti-MHC-I-AF647 (BioLegend); and streptavidin-APC (eBioscience). Bovine Serum Albumin (BSA) Cohn fraction V (A4503; Sigma) which does not contain free biotin was used for Antibody-Free Magnetic Cell Sorting.

### Cell Culture

HEK 293T cells (293 Ts) were cultured in Dulbecco's Modified Eagle Medium (DMEM) supplemented with 10% Fetal Calf Serum (FCS) and 1% penicillin/streptomycin. Primary human CD4+T cells were isolated from peripheral blood by density gradient centrifugation using Lympholyte-H (Cedarlane Laboratories) followed by negative selection using the Dynabeads Untouched Human CD4 T Cells Kit (Invitrogen) according to the manufacturer's instructions. Cells were cultured in RPMI-1640 supplemented with 10% FCS and 1% penicillin/streptomycin and activated within 48 hours using Dynabeads Human T-Activator CD3/CD28 beads (Invitrogen) according to the manufacturer's instructions. Purity was assessed by flow cytometry for CD3 and CD4 and typically found to be ≧95%.

### Plasmids

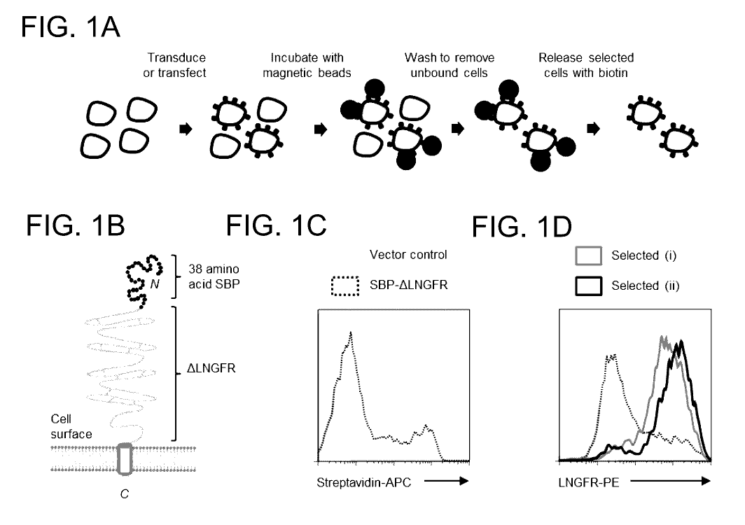

The lentiviral expression construct pHRSIN-HA-HLA-A2 (encoding HLA-A2 with an N-terminal hemagglutinin (HA) tag and a murine immunoglobulin signal peptide) has been previously described (Burr, van den Boomen et al. (2013) PNAS 108(5): 2034-2039). Overlapping DNA oligomers encoding the 38 amino acid Streptavidin Binding Peptide (SBP) (Keefe, Wilson et al. (2001) Protein Expr. Purif. 23(3): 440-446; Wilson, Keefe et al. (2001) PNAS 98(7): 3750-3755) were synthesised (Sigma), ligated and inserted using EcoRI/XhoI sites to generate pHRSIN-HA-SBP-HLA-A2. The truncated Low-affinity nerve growth factor receptor (LNGFR) was then amplified by PCR from the retroviral vector pZLRS-IRES-ΔLNGFR (Hassink, Barel et al. (2006) J. Biol. Chem. 281(4): 30063-30071) and inserted using XhoI/NotI sites in place of HLA-A2 to generate the pHRSIN-HA-SBP-ΔLNGFR construct utilised for pilot experiments in 293 Ts (FIG. 1).

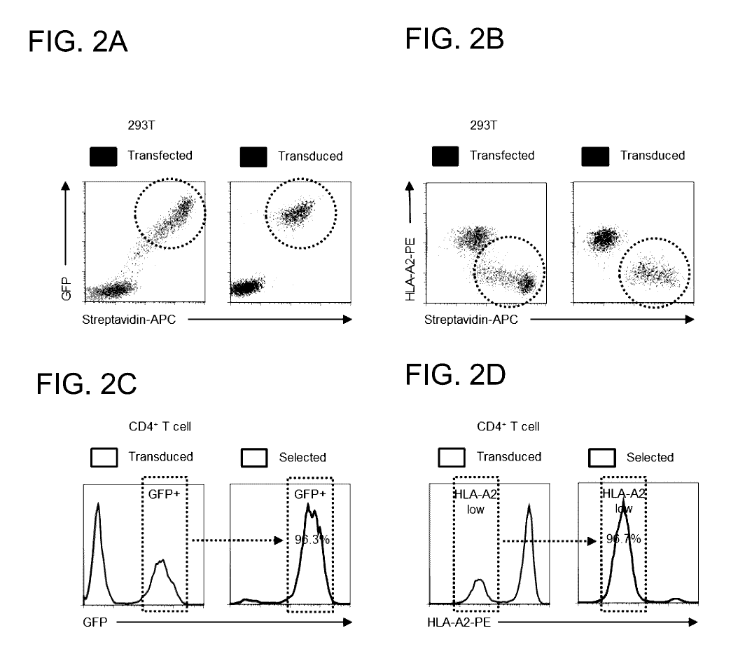

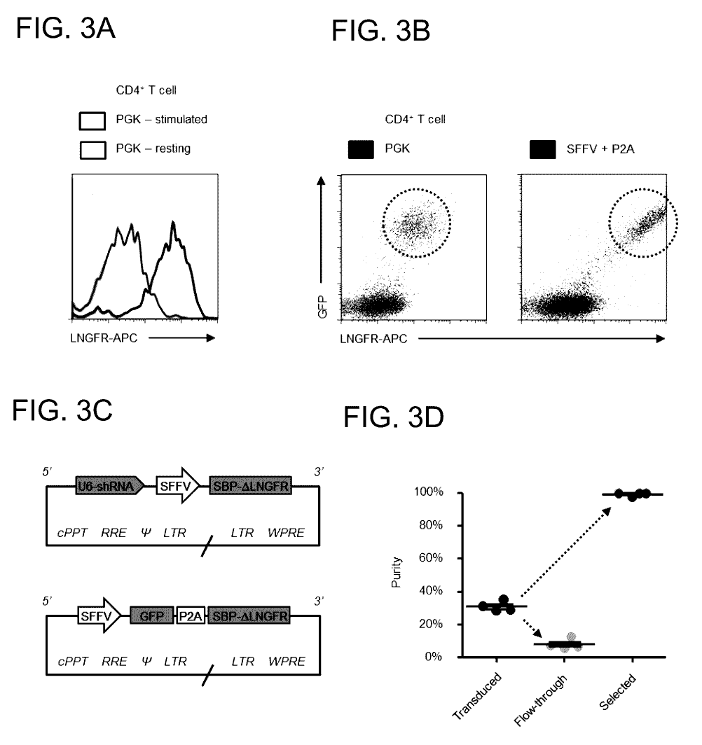

To generate bicistronic lentiviral vectors (FIGS. 2 and 3), a codon-optimised SBP-ΔLNGFR fusion protein construct was synthesised in pUC57 (Genscript). For co-expression with an exogenous gene of interest, this construct was subcloned into a self-inactivating lentiviral vector derived from pHRSIN-cPPT-SEW kindly provided by Yasuhiro Ikeda (Demaison, Parsley et al. (2002) Hum. Gene Ther. 13(7): 803-813) to generate pHRSIN-SE-PGK-SBP-ΔLNGFR-W (encoding SFFV-EGFP and PGK-SBP-ΔLNGFR with a distal Woodchuck Hepatitis Virus post-transcriptional regulatory element [WPRE]). The phosphoglycerate kinase (PGK) promoter was replaced with a Porcine teschovirus-1 2A (P2A) sequence (Kim, Lee et al. (2011) PLoS One 6(4): e18556) synthesised in pUC57 (Gencsript) to generate pHRSIN-SE-P2A-SBP-ΔLNGFR-W. BamHI and NotI sites flanking EGFP allow substitution of alternative Genes Of Interest (GOI) for co-translation as GOI-P2A-SBP-ΔLNGFR. For co-expression with an shRNA of interest, the SBP-ΔLNGFR construct was subcloned into a self-inactivating lentiviral vector derived from pCSRQ kindly provided by Greg Towers (Schaller, Ocwieja et al. (2011) PLoS Pathol. 7(12): e1002439) to generate pHRSIREN-PGK-SBP-ΔLNGFR-W (encoding a U6-shRNA cassette and PGK-SBP-ΔLNGFR with a distal WPRE). The PGK promoter was replaced with a spleen focus-forming virus (SFFV) promoter PCR-amplified from pHRSIN-cPPT-SEW to generate pHRSIREN-S-SBP-ΔLNGFR-W. BamHI and EcoRI sites allow insertion of alternative shRNAs of interest into the U6-shRNA cassette as described for the pSIREN-RetroQ vector (Clontech). For Clustered Regularly Interspaced Short Palindromic Repeats (CRISPR)/Cas9 genome editing, P2A-SBP-ΔLNGFR was subcloned from pHRSIN-SE-P2A-SBP-ΔLNGFR-W into pSpCas9(BB)-2A-Puro (PX459; Addgene) to generate pSpCas9(BB)-P2A-SBP-ΔLNGFR (encoding a U6-guide RNA (gRNA) cassette and human codon-optimized S. pyogenes Cas9 (SpCas9) co-translated with SBP-ΔLNGFR via a P2A peptide linker). BbsI sites allow insertion of site-specific gRNAs identified using the CRISPR Design Tool (http://crispr.mit.edu) (Hsu et al. (2013) Nat. Biotechnol. 31(9): 827-832) according to protocols kindly supplied by Feng Zhang (http://www.genome-engineering.org) (Cong et al. (2013) Science 339(6121): 819-823).

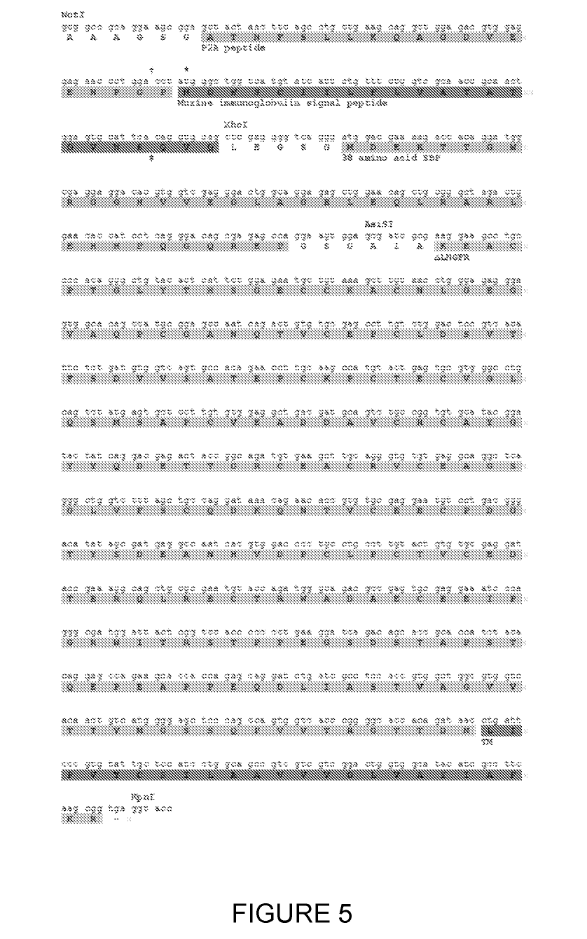

The final nucleotide and amino acid sequences of the codon-optimised SBP-ΔLNGFR construct are shown (FIG. 5). For knockdown of β2-microglobulin (β2m), the following shRNA target sequence was used: 5′-GAATGGAGAGAGAATTGAA-3′ (SEQ ID NO: 19) (Burr, van den Boomen et al. (2013) PNAS 108(5): 2034-2039). For knockout of β2m, the following gRNA target sequence was kindly selected and subcloned by Dick van den Boomen: 5′-GGCCGAGATGTCTCGCTCCG-3′ (SEQ ID NO: 20).

### Transfection and Lentiviral Transduction

FuGENE 6 (Promega; lentiviral production) or TransIT-293 (Mirus; general transfections) were used for plasmid DNA transfections in 293 Ts. To generate pseudotyped lentiviral stocks, 293 Ts were co-transfected with pHRSIN-/pHRSIREN-based lentivector, pCMVR8.91 and pMD.G, media changed at 24 hours and viral supernatant harvested and filtered (0.45 μm) at 48 hours prior to concentration using Lenti-X Concentrator (Clontech) or storage at −80° C. Transduction of primary human CD4+T cells 6-24 hours after activation was performed by spinoculation at 800 g for 1-2 hours in a benchtop centrifuge.

### Flow Cytometry

293 Ts were harvested with enzyme-free cell dissociation buffer and Dynabeads Human T-Activator CD3/CD28 beads were removed from primary human CD4+T cells using a DynaMag-2 magnet (Invitrogen). Typically 2×10⁵ washed cells were incubated for 30 minutes in 100 μL PBS with the indicated fluorochrome-conjugated antibody or streptavidin-APC. All steps were performed on ice or at 4° C. and stained cells were analysed immediately or fixed in PBS/1% paraformaldehyde.

### Antibody-Free Magnetic Cell Sorting

For pilot experiments in transfected 293 Ts (FIG. 1), washed cells were harvested with enzyme-free dissociation buffer and filtered (50 μm) to remove clumps. For selection using Dynabeads Biotin Binder (Invitrogen) cells were resuspended in Incubation Buffer (IB; PBS without calcium/magnesium, 2 mM EDTA, 0.1% BSA) at 10⁷ cells/ml and incubated with Dynabeads at a bead-to-total cell ratio of 4:1 for 30 minutes at 4° C. Bead-bound cells were selected using a DynaMag-2 (Invitrogen) then released from the beads by incubation in IB supplemented with 2 mM biotin for 15 minutes at room temperature (RT) and analysed by flow cytometry. For selection using Streptavidin MicroBeads (Miltenyi) cells were resuspended in IB at 2.5×10⁷ cells/ml and incubated with MicroBeads at a bead-to-total cell ratio of 10 μl:10⁷ cells for 30 minutes at 4° C. Bead-bound cells were selected using an MS Column and MACS Separator (Miltenyi) and analysed by flow cytometry without MicroBead removal. For selection of transduced primary human CD4+T cells, Dynabeads Human T-Activator CD3/CD28 beads were first removed according to the manufacturer's instructions. An optimised protocol for Antibody-Free Magnetic Cell Sorting using Dynabeads Biotin Binder is shown in Example 2.

### Results and Discussion

The 38 Amino Acid SBP May be Displayed at the Cell Surface by Fusion with the Truncated LNGFR.

The 38 amino acid SBP is a high-affinity streptavidin-binding peptide tag previously used for purification of recombinant proteins and, more recently, as an affinity tag in live cells for the synchronisation of secretory traffic (Keefe, Wilson et al. (2001) Protein Expr. Purif. 23(3): 440-446; Wilson, Keefe et al. (2001) PNAS 98(7): 3750-3755; Boncompain, Divoux et al. (2012) Nat. Methods 9(5): 493-498). To express the 38 amino acid SBP at the cell surface, it was fused it to the N-terminus of the truncated LNGFR (SBP-ΔLNGFR; FIG. 1b). 293 Ts transfected with this construct were readily stained with streptavidin-APC in the absence of permeabilisation, indicating expression of SBP-ΔLNGFR at the plasma membrane and accessibility for streptavidin binding (FIG. 1c). SBP-ΔLNGFR was also readily detected using an LNGFR-specific antibody (FIG. 1d). LNGFR is a 399 amino acid Type I transmembrane cell surface glycoprotein member of the Tumour Necrosis Factor Receptor superfamily (Rogers, Beare et al. (2008) J. Biol. Regul. Homeost. Agents 22(1): 1-6). The truncated LNGFR, which lacks a cytoplasmic domain, has been previously used as a non-functional cell surface marker for antibody-based cell selection, including in vitro and in vivo for purification of transduced human lymphocytes in the setting of allogenic bone marrow transplantation (Bonini, Ferrari et al. (1997) Science 276(5319): 1719-1724; Ruggieri, Aiuti et al. (1997) Hum Gene Ther. 8(13): 1611-1623). The level of cell surface streptavidin-binding peptide expression achieved was critically dependent on the fusion protein chosen, since preliminary experiments using the 38 amino acid SBP fused to the HLA-A2 heavy chain, or the streptavidin-binding Nano-tag peptide fused to a membrane-targeted red fluorescent protein construct (Lamla and Erdmann (2004) Protein Expr. Purif. 33(1): 39-47; Winnard, Kluth et al. (2007) Cancer Biol. Ther. 6(12): 1889-1899), showed poor staining at the surface of transfected cells.

### Cells Expressing SBP-ΔLNGFR May be Selected Using Streptavidin-Conjugated Magnetic Beads.

To test whether SBP-ΔLNGFR could be used for cell selection, transfected 293 Ts were incubated with streptavidin-conjugated magnetic beads. Bead-bound cells were washed, and then either analysed directly by flow cytometry, or released from the beads by incubation with excess biotin. Selected cells were markedly enriched for SBP-ΔLNGFR expression, and comparable results were achieved using streptavidin-conjugated beads from 2 different manufacturers (FIG. 1d). Dynabeads Biotin Binder were used for subsequent experiments at an optimised bead-to-target cell ratio of 10:1. Although the 38 amino acid SBP interacts strongly with streptavidin (nanomolar Kd, comparable to a strong antibody-antigen interaction), it is readily out-competed by biotin (femtomolar Kd, one of the strongest non-covalent interactions known) (Green (1990) Methods Enzymol. 184: 51-67; Brent (2001) Curr. Protoc. Protein Sci. Chapter 19: Unit 19 11; Keefe, Wilson et al. (2001) Protein Expr. Purif. 23(3): 440-446; Boncompain, Divoux et al. (2012) Nat. Methods 9(5): 493-498). In practice, bound cells could be completely released from streptavidin-conjugated beads by incubation with 2 mM biotin for as little as 15 minutes. Magnetic selection of cells expressing cell surface streptavidin (using bead-bound anti-streptavidin antibody) or co-expressing a cell surface biotin-acceptor peptide with the E. coli biotin ligase BirA (using streptavidin-conjugated beads) has been previously described (Gotoh and Matsumoto (2007) Gene 389(2): 146-153; Han, Liu et al. (2011) PLoS One 6(11): e26380; Lee and Lufkin (2012) J. Biomol. Tech. 23(2): 69-77), as has FACS of cells expressing a cell surface biotin-mimetic peptide (using fluorochrome-conjugated streptavidin) (Helman, Toister-Achituv et al. (2014) Cytometry A 85(2): 162-168). Conversely, this is the first report of the use of a cell surface streptavidin binding peptide for magnetic cell sorting, combining the advantages of bead-based cell isolation with the ability to release beads from selected cells by competition with biotin.

SBP-ΔLNGFR Affinity Purification May be Used to Isolate Cells Expressing an shRNA or Exogenous Gene of Interest.

To select genetically modified mammalian cells using SBP-ΔLNGFR affinity purification, the fusion protein was co-expressed with an exogenous gene or shRNA on the same lentiviral construct. As proof of principle, SBP-ΔLNGFR was subcloned into lentiviral vectors encoding either GFP or an shRNA to β2-microbglobulin (β2m). (β2m is an essential subunit of MHC class I molecules and its depletion may therefore be detected by reduction of cell surface MHC class I alleles such as HLA-A2 (Burr, Cano et al. (2011) PNAS 108(5): 2034-2039). Co-expression of SBP-ΔLNGFR with GFP (FIG. 2a) or shRNA to (β2m (FIG. 2b) was confirmed by transient transfection of 293 Ts, and similar results were obtained using VSVg-pseudotyped lentiviral particles (FIGS. 2a and 2b). The selection of cells genetically modified ex vivo remains a significant methodological challenge for human gene therapy. As well as the treatment of monogenic disorders such as ADA-SCID (adenosine deaminase deficiency resulting in severe combined immunodeficiency) major research efforts have focussed on cancer immunotherapy using engineered T cells expressing tumour-specific T cell receptor α and β chains (αβTCRs) or chimeric antigen receptors (CARs), and the production of HIV-resistant CD4+T cells through, for example, disruption or downregulation of the CCR5 HIV co-receptor (Kalos and June (2013) Immunity 39(1): 49-60; Kaufmann, Buning et al. (2013) EMBO Mol. Med. 5(11): 1642-1661; Peterson, Younan et al. (2013) Gene Ther. 20(7): 695-702). It was therefore tested whether magnetic selection for SBP-ΔLNGFR could be used to purify genetically modified primary human CD4+T lymphocytes expressing an exogenous gene or shRNA of interest. Indeed, following lentiviral transduction and SBP-ΔLNGFR affinity purification, pure populations of cells either high in GFP or low in HLA-A2 were successfully isolated (FIGS. 2c and 2d).

Antibody-Free Magnetic Cell Sorting Yields Greater than 99% Pure Populations of Primary Human CD4+T Cells in Less than 1 Hour.

Expression of SBP-ΔLNGFR from the PGK promoter was noted to vary markedly according to the activation state of transduced T cells (FIG. 3a). PGK encodes the glycolytic enzyme phoshpoglycerokinase, and glycolysis is known to be highly regulated in T cells (MacIver, Michalek et al. (2013) Annu. Rev. Immunol. 31: 259-283). To optimise the system for selecting primary human lymphocytes, the SFFV promoter was introduced to drive expression of SBP-ΔLNGFR either as a single cistron (pHRSIREN-S-SBP-ΔLNGFR-W) or co-translated with an exogenous gene of interest via a P2A “self-cleaving” peptide linker for bicistronic expression (pHRSIN-SE-P2A-SBP-ΔLNGFR-W). These modifications increased SBP-ΔLNGFR expression without compromising levels of the co-expressed gene or shRNA of interest (FIG. 3b). Expression levels could be increased depending on both the WPRE and the promoter strategy used, with inferior results obtained using the EF1a promoter, ECMV IRES or dual SFFV promoter systems, or when the WPRE was absent or alternatively located. The SFFV promoter is known to provide high-level transgene expression in primary human haematopoietic cells (Demaison, Parsley et al. (2002) Hum. Gene Ther. 13(7): 803-813) and 2A peptides have been shown to enable stoichiometric co-expression of multiple cistrons across different organisms and cell types (Szymczak, Workman et al. (2004) Nat. Biotechnol. 22(5): 589-594; Kim, Lee et al. (2011) PLoS One 6(4): e18556). These small viral peptide sequences are co-translationally “cleaved” in a process known as “ribosomal skipping” in which formation of a glycyl-prolyl peptide bond at the C-terminus of the 2A peptide is “skipped” without interrupting translation of the downstream polypeptide (Donnelly, Hughes et al. (2001) J. Gen. Virol. 82(Pt.5): 1027-1041). To test the optimised vectors (FIG. 3c), primary human CD4+T cells were transduced using 4 different constructs (expressing 2 different shRNAs and 2 different exogenous genes). From a starting purity of 31.0%, the average purity of selected cells was 99.2% (FIG. 3d). No deficit in cell viability or function in a wide range of downstream applications has been observed, and the Antibody-Free Magnetic Cell Sorting procedure (from incubation with magnetic beads through release with biotin) may be readily completed (including multiple samples) in less than 1 hour.

### Antibody-Free Magnetic Cell Sorting Allows Isolation of Cells Following CRISPR/Cas9 Genome Editing.

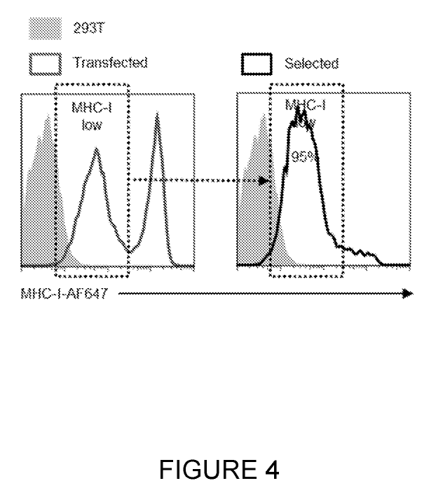

The type II bacterial CRISPR “immune system” has recently been re-purposed to allow facile site-specific genome engineering in mammalian cells by co-expression of the Cas9 nuclease with a short gRNA (Cho et al. (2013) Nat. Biotechnol. 31(3): 230-232; Cong et al. (2013) Science 339(6121): 819-823; Mali et al. (2013) Science 339(6121): 823-826). Complementary base-pairing through the gRNA recruits the gRNA/Cas9 complex to target sequences in the genomic DNA, where it introduces double-strand DNA breaks. Repair of these breaks by non-homologous end joining frequently introduces short insertions and deletions (InDels), leading to frameshifts and/or premature stop codons in the open reading frame (ORF) of the targeted gene (knock-out). Alternatively, where an exogenous DNA repair template is also supplied, homology-directed repair copies the sequence of this template to the cut target sequence, allowing the introduction of specific nucleotide changes (knock-in). To test whether Antibody-Free Magnetic Cell Sorting could be used to select cells following CRISPR gene editing, 293 Ts were transfected with a vector encoding a gRNA targeting the 5′ end of the β2m gene and SBP-ΔLNGFR co-translated with Cas9 via a P2A peptide linker (FIG. 4). As with shRNA knockdown, disruption of the β2m gene may be detected by reduction in cell surface MHC class I (MHC-I). Following Antibody-Free Magnetic Selection, MHC-I low cells were markedly enriched.

### Conclusions

Antibody-Free Magnetic Cell Sorting is a novel, efficient way to select transfected or transduced mammalian cells. Selection is readily scalable to almost any cell number and may be completed in less than 1 hour (plus cell washes). No antibody is required, allowing rapid one-step affinity purification and making the process extremely cost-effective. Enrichment to greater than 99% purity is routinely achieved and, following release with biotin, cells are left “untouched” by residual beads or antibody-antigen complexes. As well as providing a useful tool for life sciences research, the system may be used to select genetically modified cells for human gene therapy applications. Genetic modifications need not be limited to expression of shRNAs, exogenous genes of interest or CRISPR/Cas9 genome editing. For example, vectors may be developed for one-step magnetic selection of cells infected with an HIV reporter virus (Zhang, Zhou et al. (2004) J. Virol. 78(4): 1718-1729), or expression of SBP-ΔLNGFR may be used as a reporter gene for selection of cells in which a promoter of interest is active in vitro or in vivo.

### Example 2: Protocol for Antibody-Free Magnetic Cell Sorting

The following protocol has been optimised for Antibody-Free Magnetic Cell Sorting of transduced primary human CD4⁺ T cells to maximum purity using Dynabeads Biotin Binder. It may be readily scaled for almost any cell number and adapted for other transfected or transduced cell types. It is important to note that:
Adherent cells must be harvested with enzyme-free dissociation buffer
All cells must be washed thoroughly to avoid carry-over of biotin from culture media
Where indicated by the manufacturer, streptavidin-conjugated beads must be washed before use to remove preservative and/or free (unconjugated) streptavidin

### Materials

| | |
|---|---|
| Incubation Buffer (IB) | PBS without calcium/magnesium, pH 7.4 |
| Pre-cool on ice | 2 mM EDTA |
| | 0.1% BSA (A4503; Sigma) |
| Release Buffer (RB) | Complete media e.g. RPMI-1640 |
| Pre-warm to 37° C. | with 10% FCS and 1% |
| | pencillin/streptomycin |
| | 10 mM HEPES buffer, pH 7.4 |
| | 2 mM biotin |

### Protocol

1. If required, remove Dynabeads Human T-Activator CD3/CD28 beads (Invitrogen) according to the manufacturer's instructions.
2. Wash cells 3 times with cold IB then resuspend in same at 10⁷ cells/ml.
3. Add Dynabeads Biotin Binder at a bead:transduced cell ratio of 10:1 and incubate at 4° C. for 30 minutes with gentle agitation.
4. Place tube on appropriate magnet for 2-3 minutes and remove supernatant containing unbound cells.
5. Gently wash bead-bound cells once with cold IB then return to magnet for 2-3 minutes and remove supernatant containing unbound cells.
6. Resuspend bead-bound cells in pre-warmed RB at no more than 10⁷ cells/ml and incubate at room temperature for 15 minutes with gentle agitation.
7. Place tube on appropriate magnet for 2-3 minutes then transfer supernatant containing released cells to new tube.
8. If desired, to maximise yield, wash beads once with RB then return to magnet for 2-3 minutes and pool supernatants containing released cells.
9. Wash released cells twice with complete media and use as required for downstream applications.

1. A method of cell selection comprising:
(a) transfecting or transducing a cell with a nucleic acid molecule comprising a first nucleic acid sequence encoding a streptavidin binding peptide and a second nucleic acid sequence encoding a cell surface protein;
(b) expressing the nucleic acid in the cell;
(c) isolating the cell using streptavidin linked to a solid matrix; and
(d) removing the cell from the solid matrix using biotin.

2. The method as defined in claim 1, wherein the solid matrix comprises magnetic beads.

3. The method as defined in claim 1, wherein the first nucleic acid sequence encodes the Streptavidin Binding Peptide of SEQ ID NO: 1.

4. The method as defined in claim 1, wherein the cell surface protein is selected from: Low-Affinity Nerve Growth Factor Receptor (LNGFR), CD4, H-2K, CherryPicker™ or phOx sFv, such as LNGFR.

5. The method as defined in claim 1, wherein the cell surface protein is a non-functional protein.

6. The method as defined in claim 5, wherein the non-functional protein comprises a sequence of SEQ ID NO: 13.

7. The method as defined in claim 1, wherein the nucleic acid molecule encodes the amino acid sequence of SEQ ID NO: 14.

8. The method as defined in claim 1, wherein the nucleic acid molecule additionally comprises a promoter.

9. The method as defined in claim 8, wherein the promoter is a SFFV or PGK promoter.

10. The method as defined in claim 1, wherein the nucleic acid molecule is inserted into the cell genome under a native promoter.

11. A nucleic acid molecule comprising a first nucleic acid sequence encoding the Streptavidin Binding Peptide of SEQ ID NO: 1 and a second nucleic acid sequence encoding a cell surface protein.

12. A vector comprising a nucleic acid molecule as defined in claim 11.

13. The vector as defined in claim 12, which comprises a viral vector, such as a lentivirus vector.

14. The vector as defined in claim 12, which additionally comprises restriction enzyme sites suitable for insertion of a target gene.

15. A host cell which contains the vector as defined in claim 12.

16. The host cell as defined in claim 15, which is a mammalian cell, such as a human embryo kidney (HEK) cell or human T cell.

17. The host cell as defined in claim 15, which is a non-mammalian cell, such as a yeast, insect or plant cell.

18. A cell selection kit comprising the vector as defined in claim 12 and optionally together with instructions to use said kit in accordance with the method as defined in any one of claims 1 to 10.

19. The kit as defined in claim 18, which additionally comprises one or more components selected from: streptavidin coated magnetic beads, biotin, release buffer and wash buffer, such as incubation buffer.

20. Use of a kit for selecting cells comprising a vector as defined in claim 12.


SEQUENCE LISTING (20 sequences)

SEQ ID NO: 1 - protein, 38 aa, Artificial
  Synthetic Peptide
MDEKTTGWRGGHVVEGLAGELEQLRARLEHHPQGQREP

SEQ ID NO: 2 - DNA, 114 bp, Artificial
  Synthetic Nucleotide
atggacgaaaagaccacaggatggcgaggaggacacgtggtcgagggactggcaggagagctggaacagctgcgggctagactggaacaccatcctcagggacagcgagagcca

SEQ ID NO: 3 - protein, 15 aa, Artificial
  Synthetic Peptide
DVEAWLDERVPLVET

SEQ ID NO: 4 - protein, 15 aa, Artificial
  Synthetic Peptide
DVEAWLGERVPLVET

SEQ ID NO: 5 - protein, 15 aa, Artificial
  Synthetic Peptide
DVEAWLGARVPLVET

SEQ ID NO: 6 - protein, 9 aa, Artificial
  Synthetic Peptide
DVEAWLDER

SEQ ID NO: 7 - protein, 9 aa, Artificial
  Synthetic Peptide
DVEAWLGER

SEQ ID NO: 8 - protein, 9 aa, Artificial
  Synthetic Peptide
DVEAWLGAR

SEQ ID NO: 9 - protein, 101 aa, Artificial
  Synthetic Peptide
MDEKTHCFHPGDHLVRLVEELQALAEGLQRQGGRQPHRLPRRRPHHLQLLLDEAHPQAGPLRERAHQVDGRLLLQHHPQGDRLLQQPQDHPLELVWRLPPS

SEQ ID NO: 10 - protein, 101 aa, Artificial
  Synthetic Peptide
MDEKTHCTISMNGAVPLVPHHHPQGDPLRLLHRPQPALLVRHPQGDLVALVEHHEGVDRGLVALPELHAEELGEPVGDLVQGPVEQVQGVVDALVWRLPPS

SEQ ID NO: 11 - protein, 102 aa, Artificial
  Synthetic Peptide
MDEKTHWVNVYHPQGDLLVRGHGHDVEALHDQGLHQLDLLVGPPPEVVRALRGEVLGGLRRLVPLDHPQGEALDQARQRPQHLLELHHRALPPALVWRLPPS

SEQ ID NO: 12 - protein, 101 aa, Artificial
  Synthetic Peptide
MDEKTHWLEDLKGVLKDCLKDLMDFTKDCRSPRVQPQPLLHHDRGEPVPLLREAGRDLGGLGPRAPRQARPLHHGRHDLHEPLVLQDHPQGGPLVCGCHHH

SEQ ID NO: 13 - protein, 246 aa, Artificial
  Synthetic Peptide
KEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKR

SEQ ID NO: 14 - protein, 290 aa, Artificial
  Synthetic Peptide
MDEKTTGWRGGHVVEGLAGELEQLRARLEHHPQGQREPGSGAIAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKR

SEQ ID NO: 15 - protein, 22 aa, Artificial
  Synthetic Peptide
MGWSCIILFLVATATGVHSQVQ

SEQ ID NO: 16 - protein, 317 aa, Artificial
  Synthetic Peptide
MGWSCIILFLVATATGVHSQVQLEGSGMDEKTTGWRGGHVVEGLAGELEQLRARLEHHPQGQREPGSGAIAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKR

SEQ ID NO: 17 - protein, 342 aa, Artificial
  Synthetic Peptide
AAAGSGATNFSLLKQAGDVEENPGPMGWSCIILFLVATATGVHSQVQLEGSGMDEKTTGWRGGHVVEGLAGELEQLRARLEHHPQGQREPGSGAIAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKR

SEQ ID NO: 18 - protein, 298 aa, Artificial
  Synthetic Peptide
QVQLEGSGMDEKTTGWRGGHVVEGLAGELEQLRARLEHHPQGQREPGSGAIAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKR

SEQ ID NO: 19 - DNA, 19 bp, Artificial
  Synthetic Nucleotide
gaatggagagagaattgaa

SEQ ID NO: 20 - DNA, 20 bp, Artificial
  Synthetic Nucleotide
ggccgagatgtctcgctccg